(19) United States
(12) Patent Application Publication
(10) Pub. No.: US 2007/0095394 A1
(43) Pub. Date: May 3, 2007

(54) Spout assembly for endoscope reprocessor

(75) Inventors: Eric J. Deprey, Minneapolis, MN (US); Gregory Stuart Whiteley, Queenscliff (AU)

Correspondence Address: FAEGRE & BENSON;ATTN: PATENT DOCKETING, 2200 WELLS FARGO CENTER, 90 SOUTH SEVENTH STREET, MINNEAPOLIS, MN, 55402-3901, US

(73) Assignee: Minntech Corporation, Minneapolis, MN (US)

(21) Appl. No.: 11/264,902

(22) Filed: Nov. 2, 2005

(51) Int. Cl. F16K 24/00

(52) U.S. Cl. 137/216

(57) ABSTRACT

Apparatus to prevent pollution of potable water by preventing backflow in an endoscope reprocessor including a spout a spout discharging water vertically downward and a flow diverter positioned under and aligned with the spout redirecting the water off of vertical. The apparatus may also include one or more passageways either internal to a base or in external auxiliary spouts for discharging other liquids into the water in the region of the flow diverter to mnix the other liquids and water together. The apparatus may also have an air gap with a predetermined minimum length between the spout and the flow diverter or auxiliary spouts to prevent backflow.

### BACKGROUND OF THE INVENTION

The present invention relates to the field of Automatic Endoscope Reprocessors (hereinafter AERs) and similar equipment having similar characteristics and requirements.

Such equipment often is connected to a shared or community potable water supply to provide the AER with water for washing, rinsing, and diluting liquid chemicals for disinfecting endoscopes during reprocessing.

The present invention relates more particularly to water and disinfectant delivery to one or more basins of the reprocessor. In the past, various forms of spouts or other water delivery devices were used to deliver potable water from a permanently connected public water supply.

To protect the sanitary integrity of the public water supply, governmental units have promulgated various standards which must be met by equipment (including endoscope reprocessors) sold for use within the territory under the supervision of that respective governmental unit. One such standard is European Standard EN 1717:2000, Protection against pollution of potable water in water installations and general requirements of devices to prevent pollution by backflow. This European Standard is given the status of a national standard by those European countries which either publish an identical text or endorse the European Standard itself.

### SUMMARY OF THE INVENTION

The present invention meets the requirements of a type AA unrestricted air gap under the above standard, and in addition, enables mixing of water with one or more other chemical components (typically liquid) of a disinfectant solution used in the basin of an endoscope reprocessor.

The present invention may include an apparatus for delivering water to an endoscope reprocessing basin while avoiding interference with a working area of the basin. In one aspect, the invention may have at least one spout selectively discharging a liquid in a generally vertically downward direction and also has a flow diverter spaced apart from the spout by an air gap, while being positioned under the spout to receive the liquid discharged by the spout. The flow diverter then redirects the discharged liquid from the vertical direction into the basin. Stated another way, the flow diverter is a surface redirecting the discharged liquid in a direction having a horizontal component and a vertical component.

The air gap preferably has a length equal to a predetermined minimum distance between the spout and the flow diverter. The spout preferably has a downwardly directed outlet and the flow diverter will have an upper edge. The length of the air gap may be more precisely defined to be between the outlet of the spout and the upper edge of the flow diverter.

The spout has a characteristic diameter, typically an inside diameter, and the length of the air gap is preferably equal to or greater than three times the diameter of the spout.

The invention may also include a feed pipe upstream of and in fluid communication with the spout. In this aspect, the feed pipe has a characteristic diameter, typically an inside diameter, and the length of the air gap may be equal to or greater than three times the diameter of the feed pipe.

In another aspect, the present invention may include a means for discharging a second liquid into the liquid from the spout in the region of the flow diverter such that the liquids are mixed.

In another aspect, the present invention may be seen to be a method of preventing backflow and directing a liquid into an endoscope reprocessor basin comprising the steps of discharging a liquid from a spout in a downward direction; receiving the liquid discharged from the spout in a flow diverter spaced apart from the spout; and redirecting the liquid in a predetermined direction having a substantial horizontal component.

### BRIEF DESCRIPTION OF THE DRAWINGS

FIG. 1 is a very diagrammatic view in perspective of a prior art Automatic Endoscope Reprocessor useful in the practice of the present invention.

FIG. 2 is a plan view of a prior art rack with an endoscope therein suitable for use with the apparatus of FIG. 1.

FIG. 3 is a perspective view of the basins of the AER of FIG. 1.

FIG. 4 is a top plan view of the basins of FIG. 3.

FIG. 5 is an elevation view in section taken along line 5-5 of Figure 4.

FIG. 6 is a fragmentary elevation view taken along line 6-6 of Figure 4.

FIG. 7 is a perspective view of the spout assembly of the present invention showing the discharge and deflection of liquid from the assembly.

FIG. 8 is an exploded view of the parts from FIG. 7.

FIG. 9 is a top plan view of a base useful in the practice of the present invention.

FIG. 10 is a bottom plan view of the base of FIG. 9.

FIG. 11 is a perspective view from above of the base of FIG. 9.

FIG. 12 is a perspective view from below of the base of FIG. 9.

FIG. 13 is a front elevation view of the base of FIG. 9.

FIG. 14 is a section view of the base of FIG. 9 taken along line 14-14 of FIG. 10.

FIG. 15 is a partially cut away section view of the base of FIG. 9 along line 15-15 of FIG. 10.

FIG. 16 is a side view of a spout assembly showing an alternative embodiment of the present invention.

FIG. 17 is a front elevation view of the alternate embodiment of FIG. 16.

FIG. 18 is a top plan view of the alternate embodiment of FIG. 16.

### DETAILED DESCRIPTION OF THE INVENTION

One example of a system for cleaning, disinfecting and/or drying endoscopes is shown in U.S. Pat. No. 6,641,781 B2, issued Nov. 4, 2003, and the entire contents thereof are hereby incorporated by reference.

Another example of a device and method-for cleaning and/or disinfecting endoscopes is shown in U.S. Pat. No. 6,260,560 B1, issued Jul. 17, 2001, and the entire contents thereof are hereby incorporated by reference.

Still another example of a device and method for cleaning and/or disinfecting endoscopes is shown in European Patent Application EP 0 709 056 A1, published Jan. 5, 1996, and the entire contents thereof are hereby incorporated by reference.

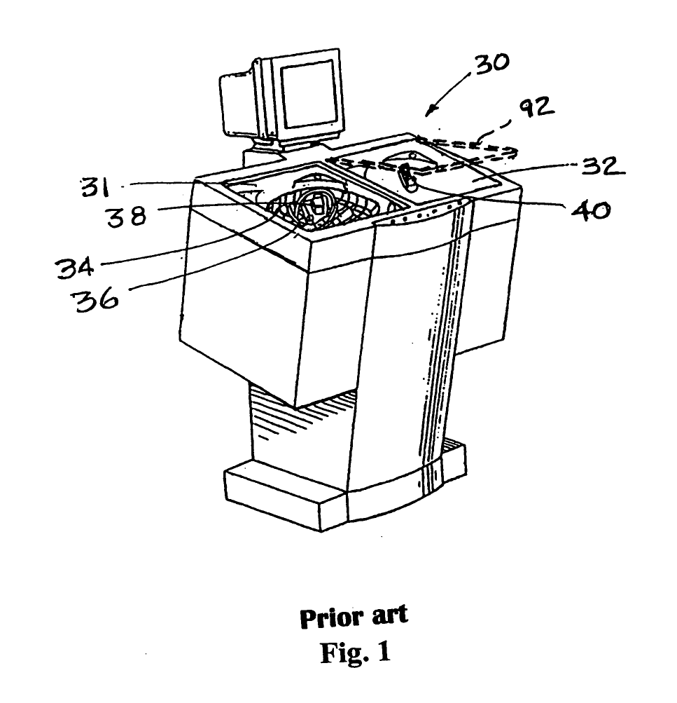

Referring now to the Figures, and most particularly to FIG. 1, a disinfecting device or Automatic Endoscope Reprocessor (or AER) 30 may be seen. The disinfecting device 30 is provided with two trays or basins 31 and 32 in which a rack 34 is, with an endoscope 36 therein, can be accommodated. In FIG. 1, a rack of this nature is located in the left hand tray. Both trays 31 and 32 are provided with a counter-connection block 40 which, when a rack 34 is placed in the tray 31, 32, can be connected to the connection block 38 arranged in rack 34. The counter-connection block 40 in the right hand tray can be seen in FIG. 1. Each tray or basin 31, 32 may be provided with a lid 92, which may be transparent.

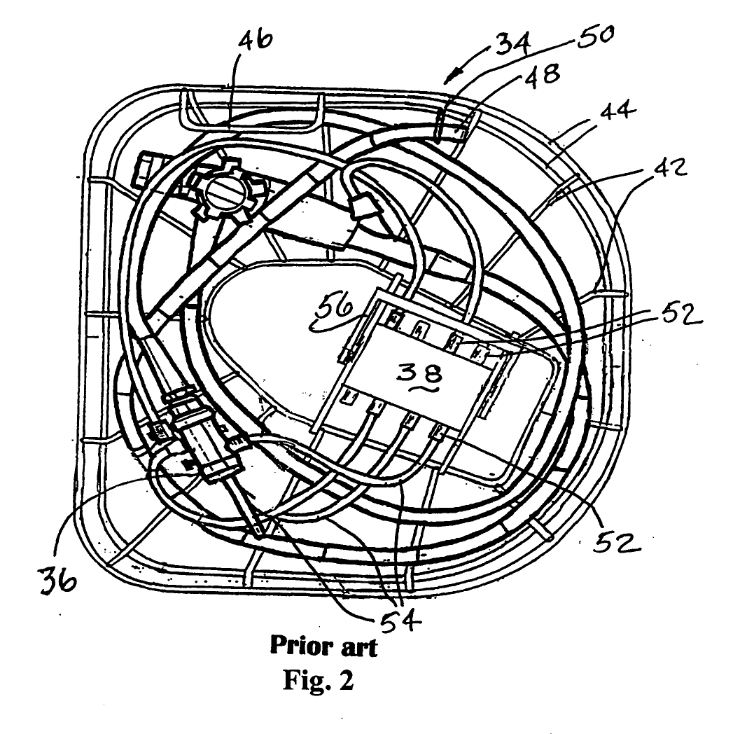

Referring now also to FIG. 2, the rack 34 may be formed from bent rods 42 and 44 which are fixedly connected to one another. The rack 34 is provided with one or two handles 46, by means of which the rack can be gripped and lifted up. The rack 34 is furthermore formed in such a manner that an endoscope 36 can be placed therein in a more or less folded state. In order to be able to fix in particular the fragile end 48 of the endoscope, the rack may be provided with a tip holder 50.

The connection block 38 is arranged fixedly in the rack. This connection block is provided with passages and ports 52 which are connected to the passages and can be connected to the passages of the endoscope 36 by means of flexible tubes 54. On its underside (not visible in FIG. 2), the connection block 38 is provided with connection points for the connection of counter-connection blocks 40 in either basin 31 or 32 of device 30. The connection block 38 is furthermore provided with a handle 56. By moving the handle 56, the connection block 38 can be connected to a counter-connection block 40 or removed therefrom.

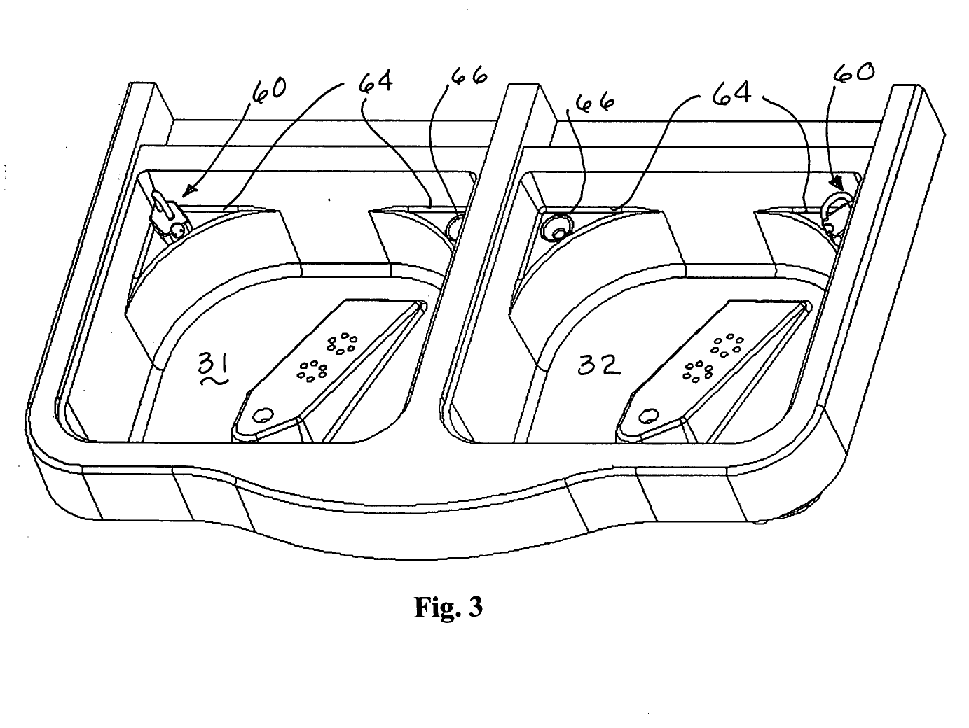

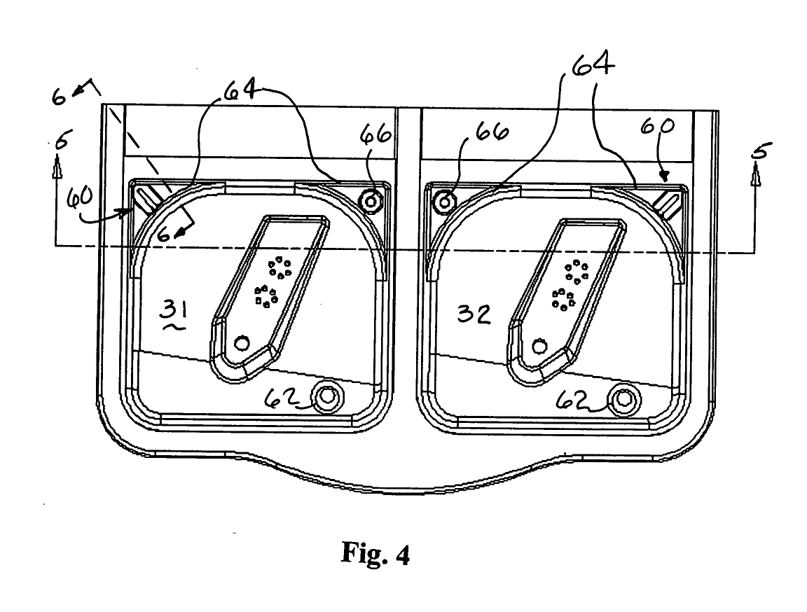

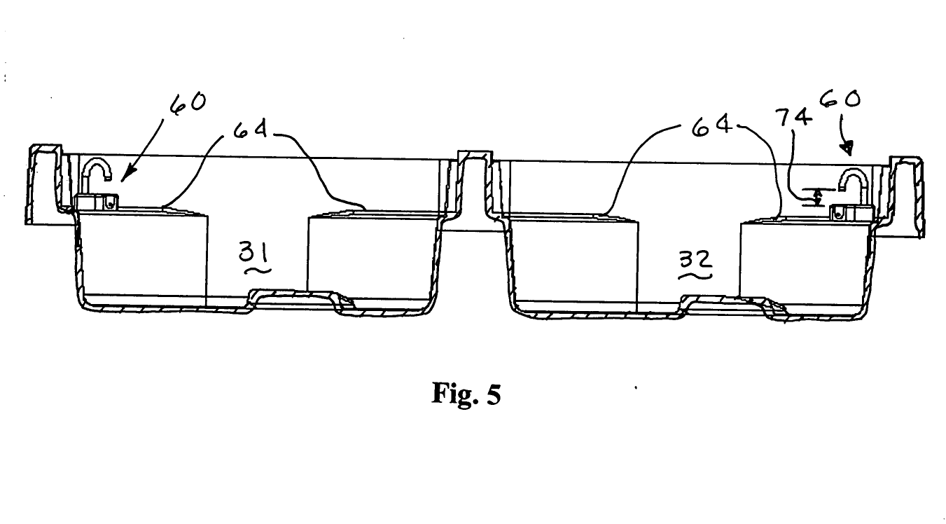

Referring now to FIGS. 3, 4 and 5, various views of the basins 31, 32 of the reprocessor 30 may be seen. Each basin preferably has a spout assembly 60 installed in one rear comer. It may be noted (particularly with reference to FIG. 4) that the spout assembly 60 provides clearance for insertion of the rack 34 into the basin 31 or 32, without interference, because the spout assembly does not extend into the periphery of the basin and overlap the area where the rack 34 would extend when lowered into the basin. Each basin has a main drain 62 near the front thereof, and a pair of shelves 64 at the rear thereof, with one shelf supporting the spout assembly 60 and the other shelf containing an overflow drain 66.

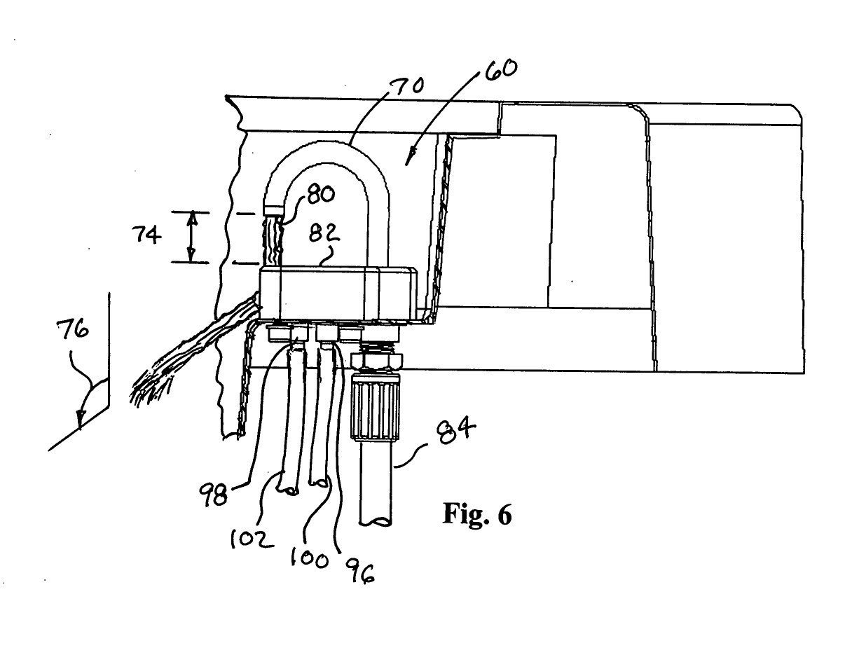

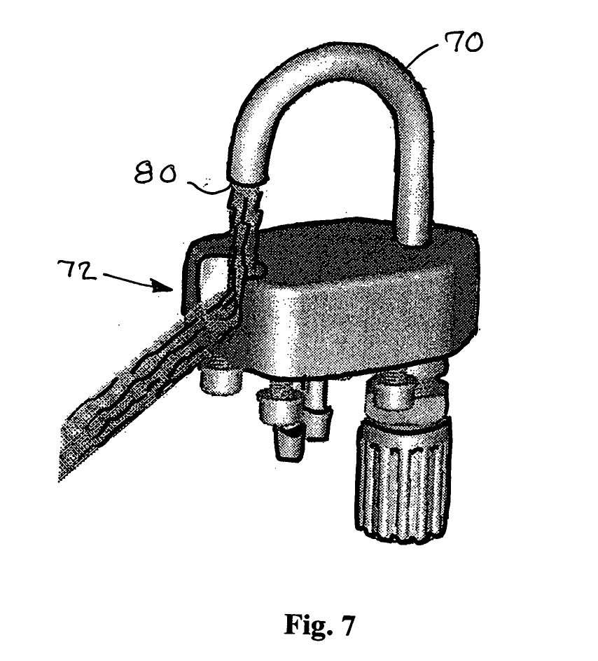

Referring now to FIGS. 6, 7 and 8, various details of the spout assembly or apparatus 60 may be seen. Spout assembly 60 preferably has a spout 70 for selectively discharging a liquid (typically potable water from a public water supply) in a generally vertically downward direction.

Spout assembly 60 also includes a flow diverter 72 spaced apart from the spout 70 by an air gap 74 and positioned under the spout and receiving the liquid discharged by the spout 70. The flow diverter 72 redirects the discharged liquid in a direction other than vertical to direct the discharged liquid into the basin. In the embodiment shown in FIGS. 6-8, the discharged liquid is initially directed at an angle 76 of about 125 degrees from the vertical, although it is to be understood that other angles are within the scope of the present invention.

In this embodiment, the flow diverter 72 is a surface 78 that may be seen to be redirecting the discharged liquid in a direction having a horizontal component and a vertical component. The air gap 74 is to be understood to have a length equal to a predetermined minimum distance between the spout and the flow diverter, with the predetermined minimum distance preferably equal to or greater than three or more diameters of the spout, more particularly either the inside or outside diameter of a discharge end or outlet 80 of the spout 70. It may thus be seen that the spout 70 has downwardly directed outlet 80 and the length of the air gap 74 is preferably measured between the outlet 80 of the spout and the flow diverter 72. More precisely, in this embodiment the flow diverter 72 is preferably formed (in this embodiment) in a base 86 which has an upper edge 82 and the length of the air gap 74 is measured between the outlet 80 of the spout 70 and the upper edge 82 of the flow diverter 72.

The spout assembly 60 is preferably connected to a feed pipe 84 upstream of and in fluid communication with the spout 70. The feed pipe 84 has a diameter, and the length of the air gap 74 may be made equal to or greater than three times the diameter of the feed pipe 84 in the practice of the present invention. Furthermore, the portion of the spout 70 upstream of the outlet 80 may be considered the feed pipe for air gap dimensioning in accordance with the practice of the present invention. In that regard, the diameter used for dimensioning the air gap may be an inside or outside diameter of the feed pipe, including spout 70. It is preferred that the direction of discharge from the outlet 80 of spout 70 is not more than fifteen degrees from the vertical.

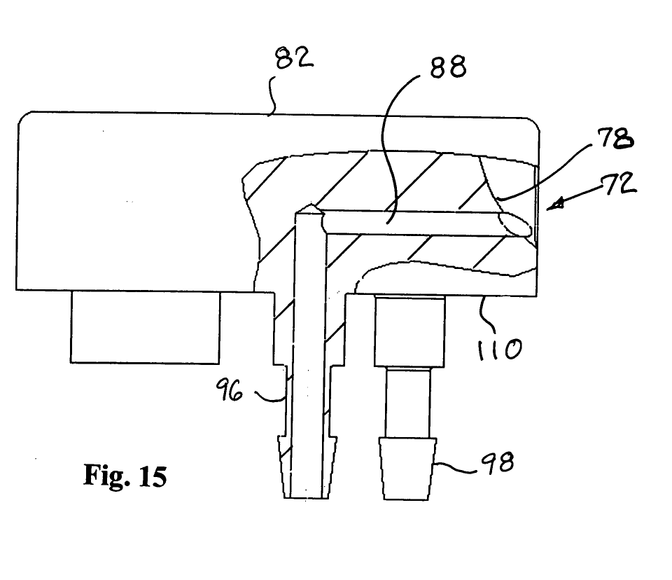

The liquid discharged from the spout 70 may be considered a first liquid and the apparatus of the present invention further includes means for discharging at least a second liquid (and preferably a third liquid) into the first liquid in the region of the flow diverter 72 such that the liquids are mixed- In the embodiment shown in FIGS. 6-8, the means for discharging the second liquid (and optionally the third liquid) is one or more internal passages 88 in the base 86 (shown in FIG. 15).

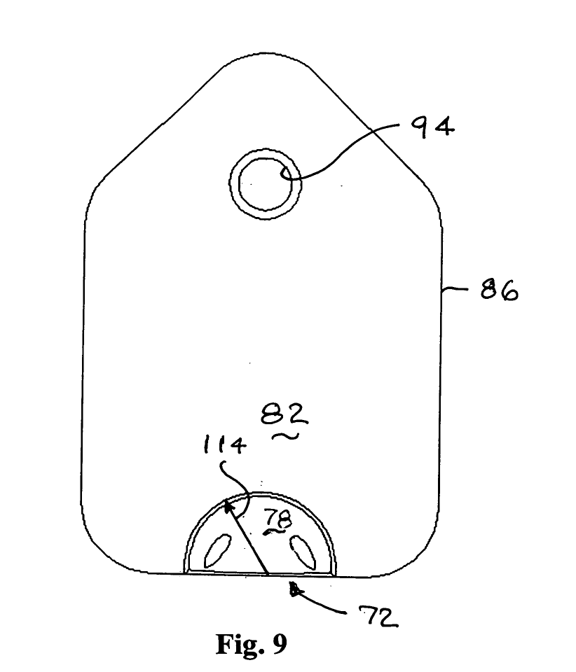

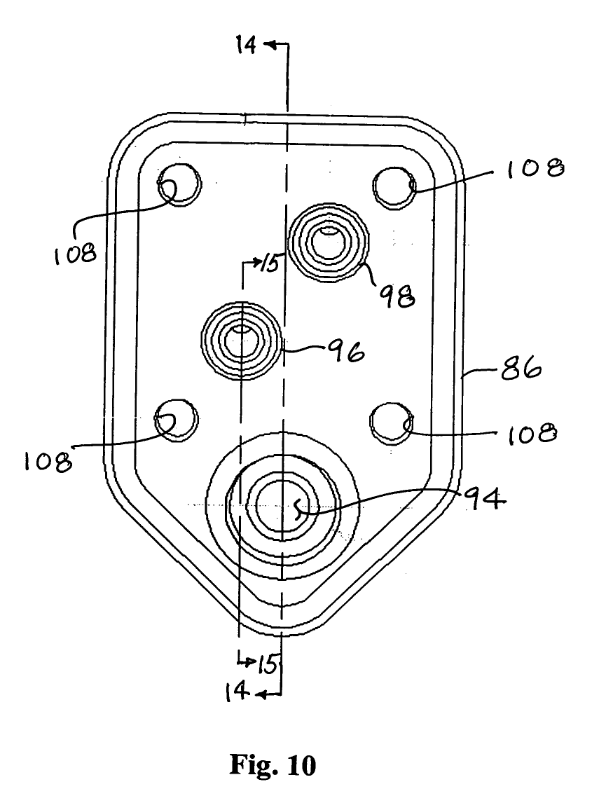

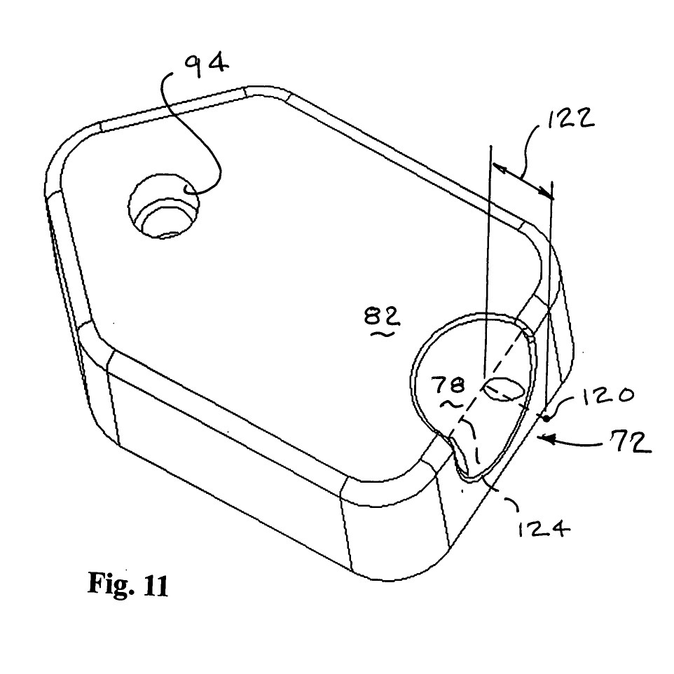

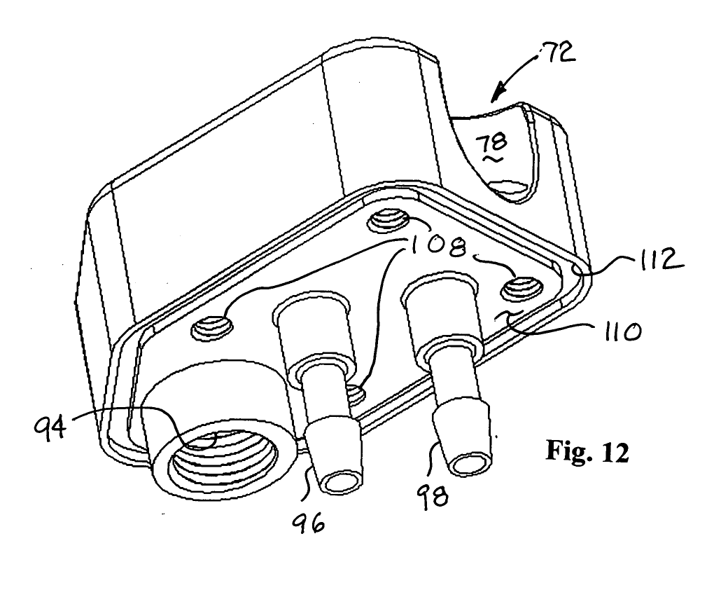

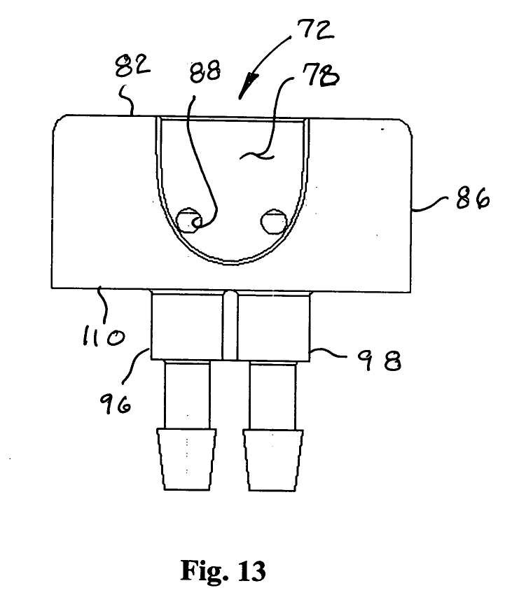

Referring now most particularly to FIGS. 9 through 15, various views of the base 86 for the spout assembly 60 of the present invention may be seen. It is to be understood that alternative embodiments for the base 86 and for the flow diverter 72 are within the scope of the present invention, and the base 86 and flow diverter 72 are shown and described as an illustration of one embodiment of the present invention. Base 86 is shown as a solid material formed of, for example 316 stainless steel, but the base 86 (and other components of the spout assembly 60) may advantageously be formed of other suitable materials, such as other metals or polymers, if desired, and the flow diverter 72 may be formed, for example, from a sheet of material, if desired.

Base 86 has a through bore 94 to permit the passage of water to spout 70. In this embodiment, base 86 also has fittings 96 and 98 to receive respective tubes 100 and 102 to couple the two liquid disinfectant chemicals to the base and to their respective internal passages 88.

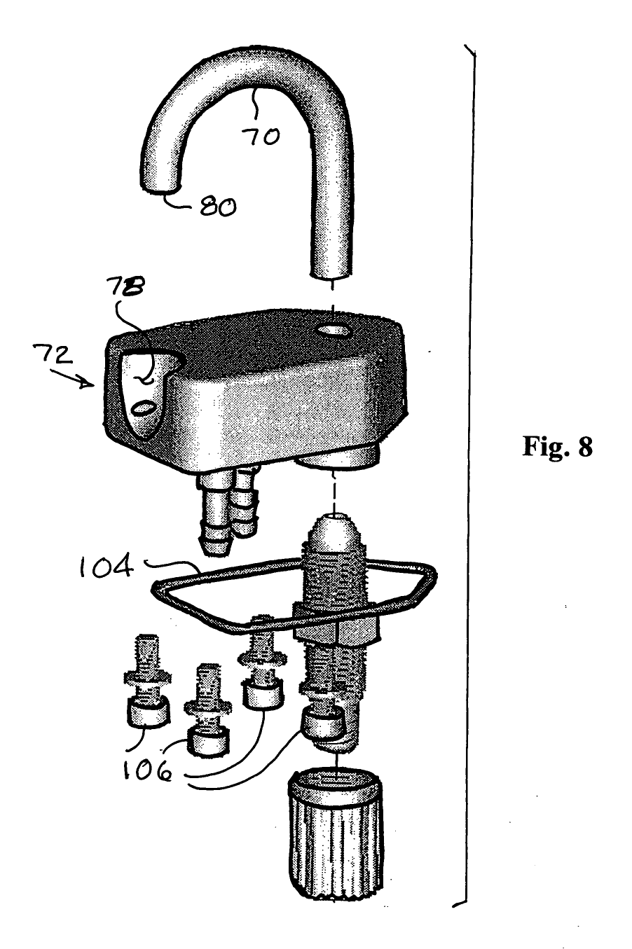

Referring now again to FIG. 8, an O-ring 104 may be used to seal the base 86 to shelf 64 and a plurality of fasteners 106, such as cap screws and washers, may be used to secure base 86 to shelf 64, by receiving the fasteners 106 into blind threaded bores 108 in the bottom surface 110 of base 86. A groove 112 may be formed in the bottom surface 110 to receive O-ring 104 or another seal may be used to seal the base to the shelf, if desired.

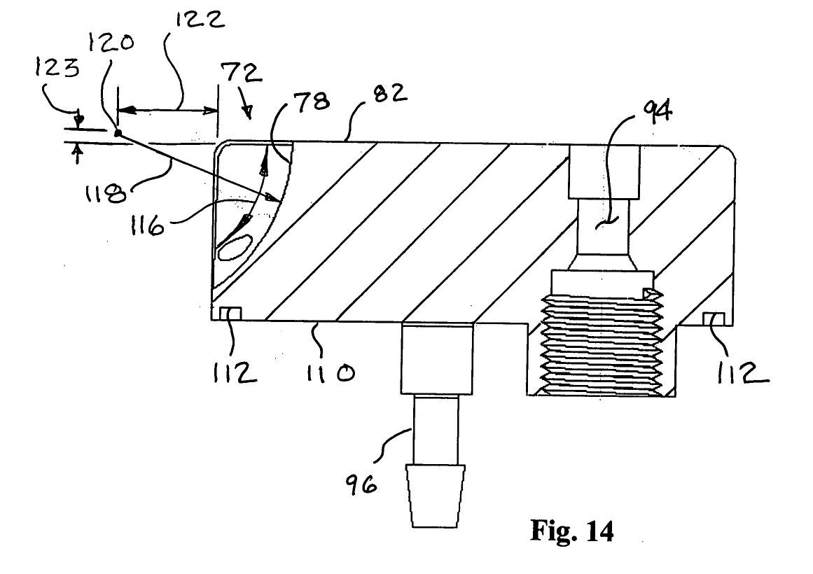

It is to be understood that the surface contour of the flow diverter 72 can take many forms while still remaining within the scope of the present invention. Referring now again to FIGS. 9, 11 and 14, one example surface 78 may be formed by a cutout having a radius 114 of 8 mm (shown in FIG. 9) which is swept through an arc 116 (shown in FIG. 14) having a radius 118 of 40 mm from a center 120 located a horizontal distance 122 of 29.92 mm and a vertical distance 123 of 2.55 mm from the front edge 124 of the envelope of top edge 82 of the base 86.

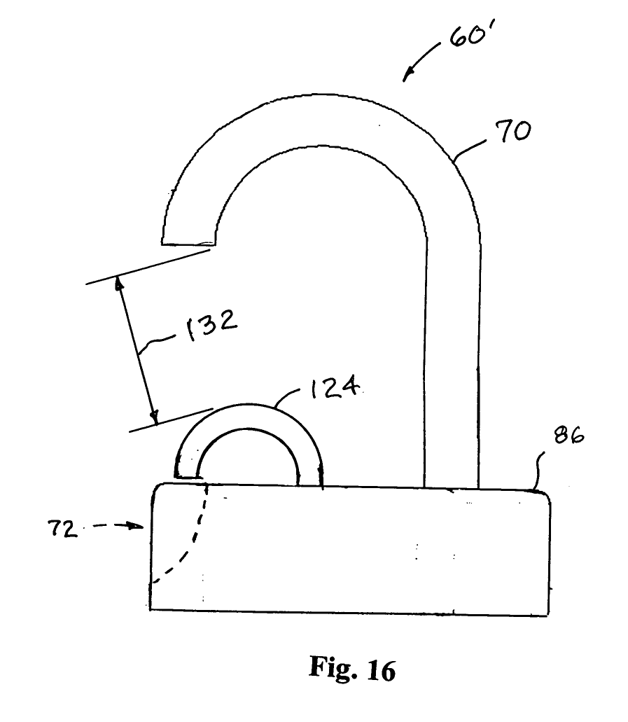

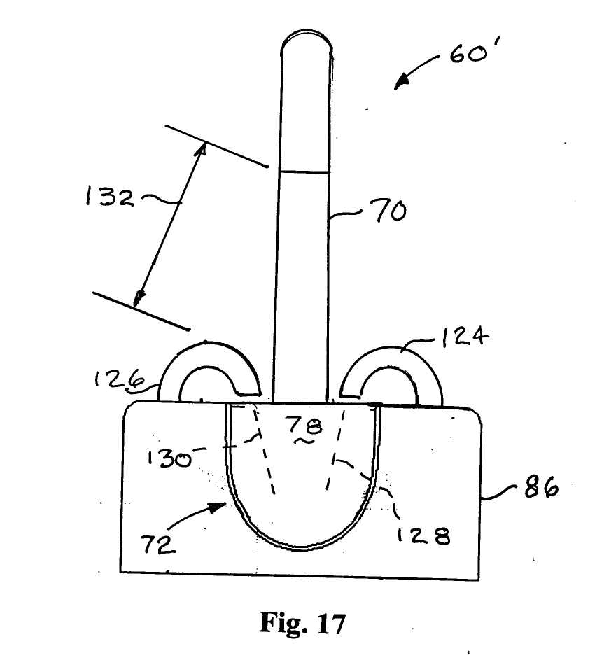

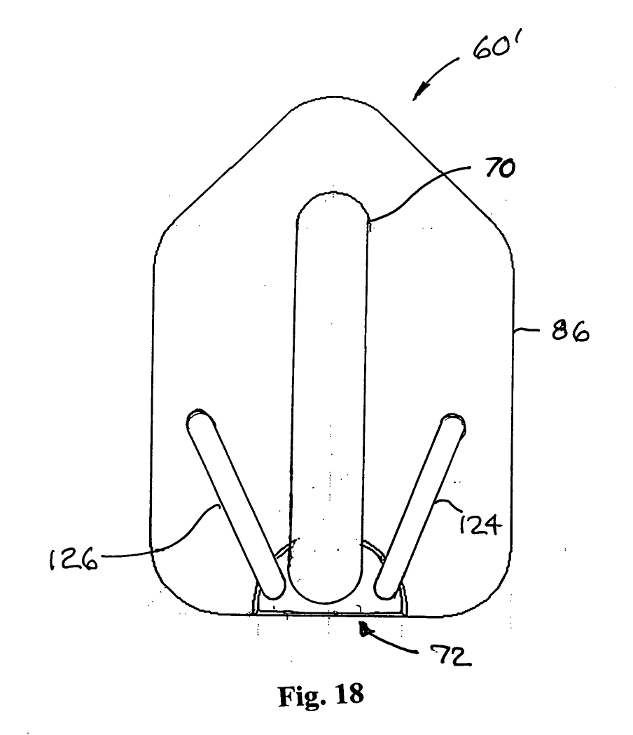

Referring now to FIGS. 16, 17 and 18, an alternative embodiment 60′ of the spout assembly of the present invention may be seen. In this embodiment, the water spout 70 is the same or similar to that in the previous embodiment, but internal passages 88 are omitted and replaced by liquid chemical delivery spouts 124, 126. The flow diverter 72 may be the same or similar to that in the previous embodiment, except that there are no apertures for the internal passages 88 in the surface 78 of the flow diverter 72 in this embodiment. The spouts 124 and 126 each have internal passages for delivery of a liquid chemical. Each of spouts 124 and 126 may be directed vertically downward or may be directed at an angle indicated by dashed lines 128, 130 to foster mixing with the stream of water from the spout 70. It is preferable in this embodiment that the chemical spouts 124 and 126 are each spaced a distance 132 equal to or greater than at least three diameters of the water spout 70 away from the water spout 70.

This invention is not to be taken as limited to all of the details thereof as modifications and variations thereof may be made without departing from the spirit or scope of the invention. In particular, the present invention as claimed is not limited for use with an automatic endoscope reprocessor (including, but not limited to, reprocessors for flexible endoscopes), but may be used with other equipment having similar characteristics or requirements.

What is claimed is:

1. Apparatus for delivering water to an endoscope reprocessing basin while avoiding interference with a working area of the basin, the apparatus comprising:
a. a spout selectively discharging a liquid in a generally vertically downward direction; and
b. a flow diverter spaced apart from the spout by an air gap and positioned under the spout and receiving the liquid discharged by the spout and redirecting the discharged liquid in a direction other than vertical.

2. The apparatus of claim 1 wherein the flow diverter is a surface redirecting the discharged liquid in a direction having a horizontal component and a vertical component.

3. The apparatus of claim 1 wherein the air gap further comprises a length equal to a predetermined minimum distance between the spout and the flow diverter.

4. The apparatus of claim 3 wherein the spout has a downwardly directed outlet and the length of the air gap is measured between the outlet of the spout and the flow diverter.

5. The apparatus of claim 4 wherein the flow diverter has an upper edge and the length of the air gap is measured between the outlet of the spout and the upper edge of the flow diverter.

6. The apparatus of claim 5 wherein the spout has a diameter and the length of the air gap is equal to or greater than three times the diameter of the spout.

7. The apparatus of claim 6 wherein the diameter is an inside diameter of the spout.

8. The apparatus of claim 5 further comprising a feed pipe upstream of and in fluid communication with the spout and wherein the feed pipe has a diameter and the length of the air gap is equal to or greater than three times the diameter of the feed pipe.

9. The apparatus of claim 8 wherein the diameter is an inside diameter of the feed pipe.

10. The apparatus of claim 1 wherein the liquid discharged from the spout comprises a first liquid and the apparatus further includes
c. means for discharging a second liquid into the first liquid in the region of the flow diverter such that the first and second liquids are mixed.

11. The apparatus of claim 10 further comprising:
d. means for discharging a third liquid into the first liquid in the region of the flow diverter such that the first, second and third liquids are mixed.

12. The apparatus of claim 1 wherein the direction of discharge of the spout is not more than fifteen degrees from the vertical.

13. A spout assembly for preventing backflow in a water supply system comprising:
a. a water spout selectively directing a stream of water generally vertically downward;
b. a flow diverter positioned vertically under and receiving the downwardly directed stream of water from the water spout and wherein the flow diverter has a concave surface redirecting the stream.

14. The spout assembly of claim 13 further including at least one passage for delivering a liquid chemical to the flow diverter to mix the at least one liquid chemical with the stream of water.

15. The spout assembly of claim 14 wherein the at least one passage exits in a surface of the flow diverter.

16. The spout assembly of claim 14 wherein the at least one passage is in at least one liquid chemical delivery spout above the flow diverter.

17. The spout assembly of claim 16 wherein the at least one chemical delivery spout emits a stream at an angle towards the stream of water.

18. The spout assembly of claim 16 wherein the at least one chemical delivery spout is spaced a distance apart from the water spout equal to or greater than three times the diameter of the water spout.

19. The spout assembly of claim 18 wherein the diameter is an inside diameter.

20. The spout assembly of claim 18 wherein the diameter is an outside diameter.

21. A method of preventing backflow and directing a liquid into an endoscope reprocessor basin comprising the steps of:
a. discharging a liquid from a spout in a downward direction;
b. receiving the liquid discharged from the spout in a flow diverter spaced apart from the spout; and
c. redirecting the liquid in a predetermined direction having a substantial horizontal component.
